(19) United States
(12) Patent Application Publication
(10) Pub. No.: US 2012/0209062 A1
(43) Pub. Date: Aug. 16, 2012

(54) MOTHER-BABY ENDOSCOPE SYSTEM CONSISTING OF HARD MOTHER-CHOLECYSTOSCOPE AND FLEXIBLE BABY-CHOLEDOCHOSCOPE

(76) Inventor: Tie Qiao, Guangzhou (CN)

(21) Appl. No.: 13/322,058

(22) PCT Filed: Jul. 15, 2010
(86) PCT No.: PCT/CN10/75167

§ 371 Date: Nov. 22, 2011

(30) Foreign Application Priority Data
Nov. 6, 2009 (CN) 200910223111.8

(51) Int. Cl. A61B 1/018; A61B 1/005; A61B 1/12; A61B 1/04; A61B 1/06

(52) U.S. Cl. 600/104

(57) ABSTRACT

The present invention relates to a medical apparatus, in particular, a mother-baby endoscope system consisting of hard mother-cholecystoscope and flexible baby-choledochoscope which comprises a multi-channel cholecystoscope utilized as a mother endoscope and a flexible choledochoscope utilized as a baby endoscope; The flexible choledochoscope comprises a bendable front end, an instrument channel connected to the front end, an operating part and a light source host machine connected to said operating part via a cable, a video camera system, and a water supply equipment is also connected to the flexible choledochoscope for water supplying. The front end of baby endoscope passes through the instrument channel of the mother endoscope. The multi-channel cholecystoscope enters into gallbladder cavity, removes the gallstones in the gallbladder cavity and gallbladder walls, then the flexible choledochoscope enters into cystic duct, common bile duct, common hepatic duct, left and right hepatic duct in porta hepatis to perform treatments.

### FIELD OF TECHNOLOGY

The present invention relates to a medical apparatus, in particular, a mother-baby endoscope system consisting of hard mother-cholecystoscope and flexible baby-choledochoscope, as well as operating method thereof, which is a core medical apparatus to carry out a mini-invasive endoscopic cholelithotomy without excision of gallbladder in modern medicine.

### BACKGROUND

Cholelithiasis is a common disease and frequently-occurring disease. The gallstone exists not only in gallbladder, but also in cystic duct, intrahepatic bile duct and extrahepatic bile duct. It is understood that, the morbidity of cholecystolithiasis complicating choledocholithiasis was 6% to 10% in total cholelithiasis in China and the treatment of which is the difficulty in biliary tract surgery. There are three treatment methods, wherein the first one is cholecystectomy and exploration of common bile duct which will be conducted by laparoscopic operation or celiotomy; The second one is cholecystectomy and ERCP which will be conducted by laparoscopic operation or celiotomy (ERCP stands for Endoscopic Retrograde CholangioPancreatography, which is an imaging technique that pancreaticobiliary duct can be displayed retrogradely after the contrast medium was injected through duodenal papilla intubation under the endoscope, ERCP is currently an accepted standard in the diagnosis of cholangiopancreatic duct diseases); The third one is endoscopic cholelithotomy without excision of gallbladder and ERCP. The disadvantage of said first and second methods is that the gallbladder must be removed. The disadvantage of said third method is that its operation is complicated, which is generally used by associating hard baby-endoscope with hard mother-endoscope. The front end of hard baby-endoscope is difficult to be placed into common bile duct, common hepatic duct and left and right hepatic duct in porta hepatis to remove the gallstones because of its inflexibility, consequently gallstones in common hepatic duct, and gallstones in primary and secondary bile ducts in porta hepatis can not be completely taken out and some postoperative complications may occur in a certain condition.

### SUMMARY

It is an object for the present invention to overcome the defect in the prior art that the cholecystolithiasis complicating choledocholithiasis were unable to be treated well. The present invention presents a mother-baby endoscope system consisting of hard mother-cholecystoscope and flexible baby-choledochoscope, as well as operating method thereof. The mother-baby endoscope system can be placed into common bile duct, common hepatic duct and left and right hepatic duct in porta hepatis, to perform the treatment such as removal of gallstones, which can completely remove the gallstones with a good therapeutic effect and low risk of postoperative complications.

With regard to the object, the present invention provides the following technical solutions that:

A mother-baby endoscope system described in the present invention comprises a multi-channel cholecystoscope utilized as mother endoscope and a flexible choledochoscope utilized as baby endoscope. The multi-channel cholecystoscope comprises a hard endoscope body, an instrument channel arranged in the hard endoscope body, an input port of an ocular, an input port of cold light source, a water inlet channel and a water outlet channel, a light source host machine disposed with said input port of cold light source of said mother endoscope, and a video camera system of input port of the ocular; Said flexible choledochoscope comprises a bendable front end, an instrument channel connected with the front end, an operating part and a light source host machine connected with said operating part via a cable, and a video camera system. A water supply equipment is also connected with said flexible choledochoscope for supplying water thereto; The front end of said baby endoscope passes through the instrument channel of said mother endoscope.

In the present invention, the apex part of front end of said flexible choledochoscope is extremely slim and has an external diameter to be within 3.0 mm (≦3.0 mm), and the length of the apex part is 500 mm. A bending portion is also disposed at the apex part of the front end, its bending angle is up to 180° in vertical direction, and up to 160° in horizontal direction, and the length of the bending portion is less than or equal to 25 mm (≦25 mm). The flexible choledochoscope can be either an electronic choledochoscope which adopts an electronic imaging optical system, or a fiber choledochoscope which adopts a fiber imaging optical system.

Furthermore, the diameter of front end of the hard endoscope body of the multi-channel cholecystoscope ranges from 6.5 mm to 7.5 mm, its length ranges from 200 mm to 220 mm. The diameter of the operating instrument channel of said multi-channel cholecystoscope is more than or equal to 3.5 mm (≧3.5 mm). The ocular input port of the multi-operation-channel cholecystoscope utilized as a mother endoscope is at the angle of 135 degree to the central axis of hard endoscope body, and which forms overall gun-shape that suits for the operation of a doctor and makes the operation stable. 2.8 mm optical lens are adopted by the optical system in the endoscope; There are three kinds of multi-channel cholecystoscopes classified by the optical lens having the field angle of 0 degree, 12 degree or 30 degree respectively.

An operating method for the mother-baby endoscope system consisting of hard mother-cholecystoscope and flexible baby-choledochoscope, as following steps:

(1) locating the position of gallbladder, placing the multi-channel cholecystoscope into the gallbladder cavity through an incision at the bottom of the patient's gallbladder, then completely removing the gallstones in the gallbladder cavity and the gallbladder walls of the patients by using the hard and flexible surgical equipments such as biopsy forceps, basket type grasping forceps, gravel bar or electro-coagulator through the instrument channel of the multi-channel cholecystoscope;

(2) confirming the entry of cystic duct by means of the multi-channel cholecystoscope, placing the flexible choledochoscope into cystic duct through the wide instrument channel of the multi-channel cholecystoscope, and taking advantage of the front end of the flexible choledochoscope with small diameter and bendability then further into the common bile duct, common hepatic duct and left and right hepatic duct in porta hepatis, simultaneously removing the gallstones by small flexible equipment such as basket type grasping forceps or biopsy forceps.

Comparing with the prior art, the benefits of the present invention are:

The present invention uses a hard endoscope and a flexible endoscope, to takes out gallstones from the gallbladder cavity and the gallbladder walls by means of the multi-channel cholecystoscope, and the flexible choledochoscope enters into the common bile duct, common hepatic duct and left and right hepatic duct in porta hepatis taking advantage of the front end of the flexible choledochoscope with small diameter and bendability, simultaneously surgical treatment such as the removal of gallstones from extra-hepatic duct and primary and secondary bile ducts in porta hepatis is performed with small flexible equipment (such as basket type grasping forceps, biopsy forceps, etc.). Such that gallstones can be taken out completely and no complications are caused by residual gallstones after the operation. The present invention can be used conveniently and reliably, and has a good therapeutic effect.

### BRIEF DESCRIPTION OF THE DRAWINGS

FIG. 1 is a general structure schematic diagram of a mother-baby endoscope system consisting of hard mother-cholecystoscope and flexible baby-choledochoscope.

FIG. 2 is a structure schematic diagram of mother endoscope, i.e. multi-operation-channel cholecystoscope, according to the present invention.

FIG. 3 is a schematic diagram of the apex part of mother endoscope, i.e. multi-operation-channel cholecystoscope, according to the present invention.

FIG. 4 is a structure schematic diagram of baby endoscope, i.e. flexible choledochoscope, according to the present invention.

FIG. 5 is a schematic diagram of bending portion of baby endoscope, i.e. flexible choledochoscope, when bending in vertical direction according to the present invention.

FIG. 6 is a schematic diagram of bending portion of baby endoscope, i.e. flexible choledochoscope, when bending in horizontal direction according to the present invention.

FIG. 7 is a schematic diagram in surgery process according to the present invention.

### DETAIL DESCRIPTION

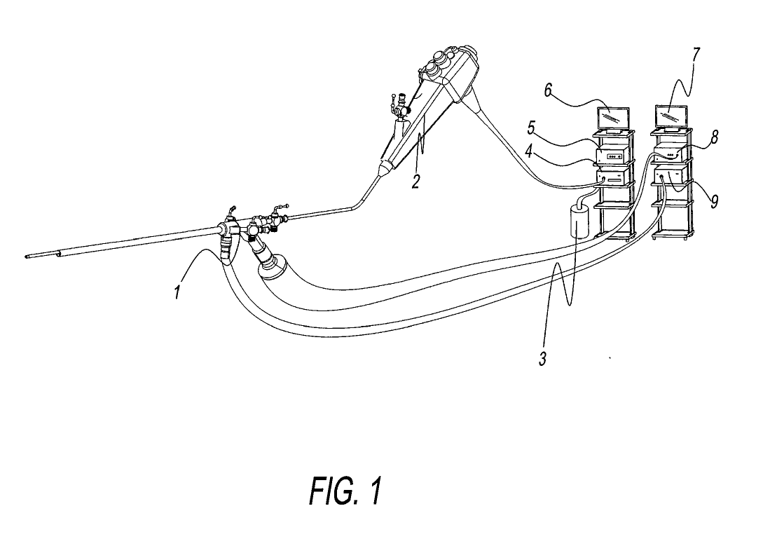

As shown in FIG. 1, the mother-baby endoscope system described in the present invention comprises a flexible choledochoscope 2 utilized as baby endoscope, a multi-operation-channel cholecystoscope 1 utilized as mother endoscope, a water jar 3 for the usage of flexible choledochoscope 2, a video camera host machine 4, a light source host machine 5, a monitor 6, a light source host machine 9 connected with multi-operation-channel cholecystoscope 1, a video camera host machine 8 and a monitor 7. FIG. 1 shows the connection mode of the mother-baby endoscope system consisting of hard mother-cholecystoscope and flexible baby-choledochoscope.

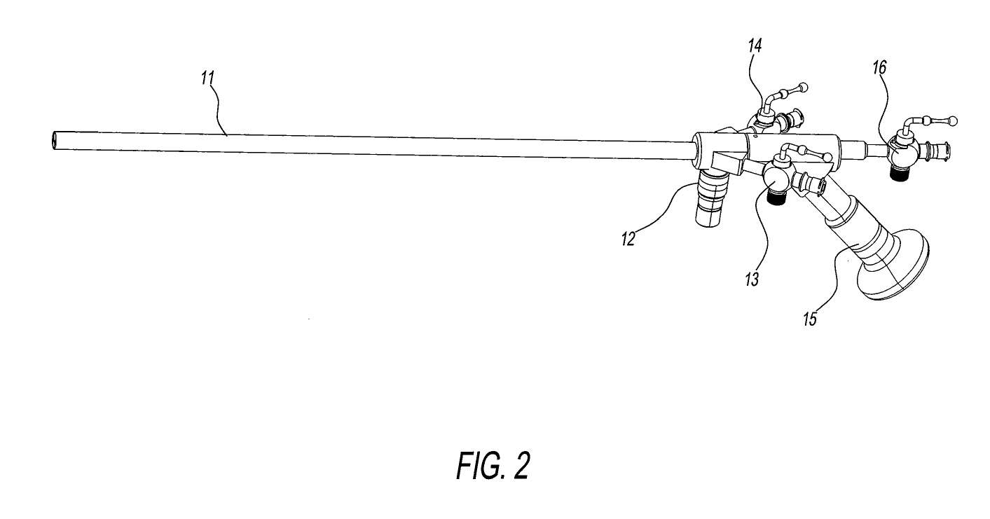

FIG. 2 shows a mother endoscope of the present invention, that is, a multi-operation-channel cholecystoscope 1 which is consisting of a front end 11 of hard endoscope body, an input port of light source 12, a water inlet channel 13, a water outlet channel 14, an input port of ocular 15 and an instrument channel 16, wherein an effective length of the front end 11 of the endoscope body is 200 to 220 mm, and its external diameter is 6.5 to 7.5 mm.

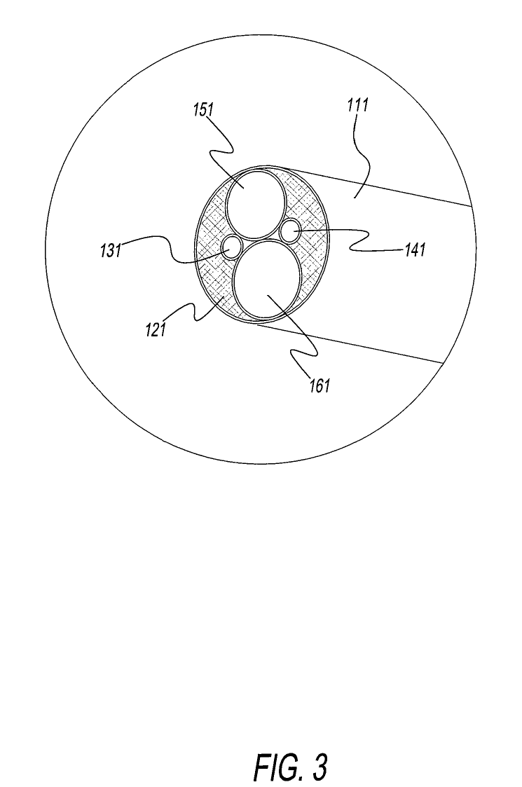

FIG. 3 is a structure schematic diagram of the apex part 111 of multi-operation-channel cholecystoscope 1. The apex part 111 comprises an optical lens 151 having a diameter of 2.8 mm and an exit of instrument channel 161 having a diameter of 3.2 mm. There are further two independent exits, i.e. an exit of the water inlet channel 131 and an exit of the water inlet channel 141. An optical fiber 121 is arranged inside the apex part 111 to provide the light resource for surgery.

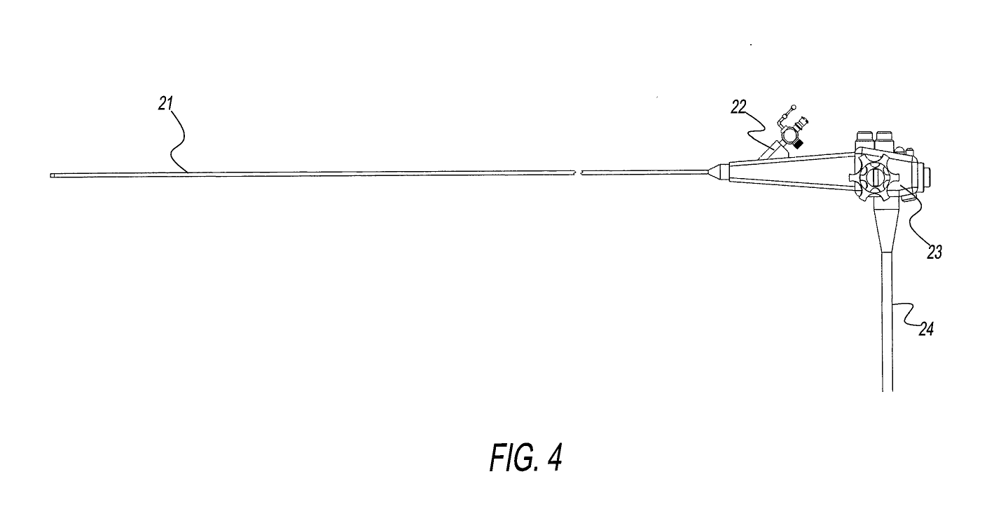

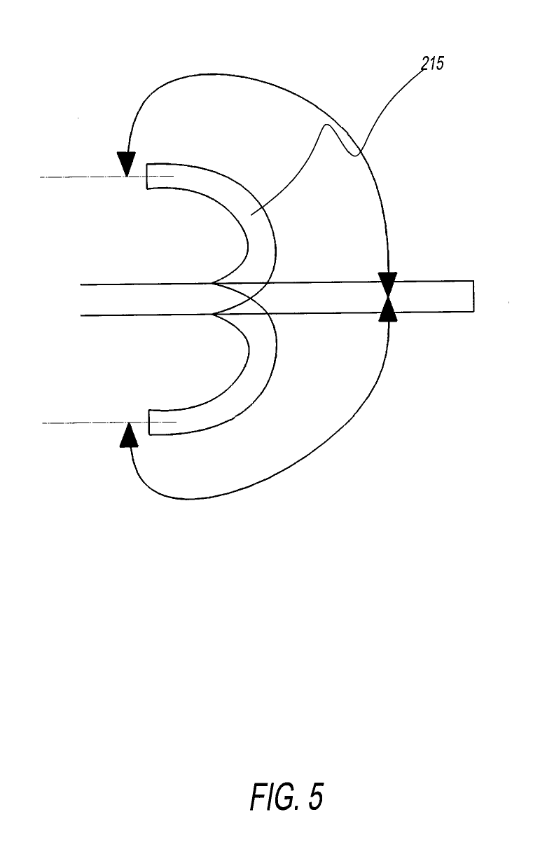

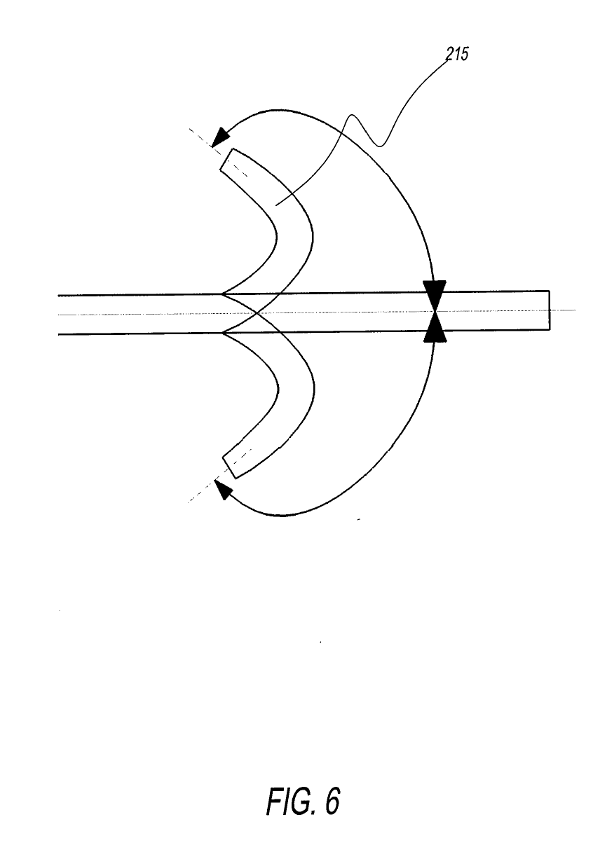

FIGS. 4 to 6 are schematic diagrams of a flexible choledochoscope 2. The flexible choledochoscope 2 comprises a front end 21, an instrument channel connector 22, an operating part 23 and a data cable 24. The flexible choledochoscope 2 can be an electronic choledochoscope basing on an electronic imaging optical system or a fiber choledochoscope basing on a fiber imaging optical system. In the present invention, the apex part of the front end 21 is extremely slim and has an external diameter to be less than or equal to 3.0 mm (≦3.0 mm), and the length of its apex part is 500 mm. Furthermore, a bending portion 215 is also disposed at the apex part of the front end 21, its bending angle is up to 180° in vertical direction, and up to 160° in horizontal direction, and the length of the bending portion 215 is less than or equal to 25 mm (≦25 mm). The bendable front end 21 of flexible choledochoscope 2 is placed into common bile duct, common hepatic duct, left and right hepatic duct in porta hepatis via said bending portion 215, which can completely remove the gallstones. The technique is practical and reliable with a high cure rate and little incidence of postoperative complications.

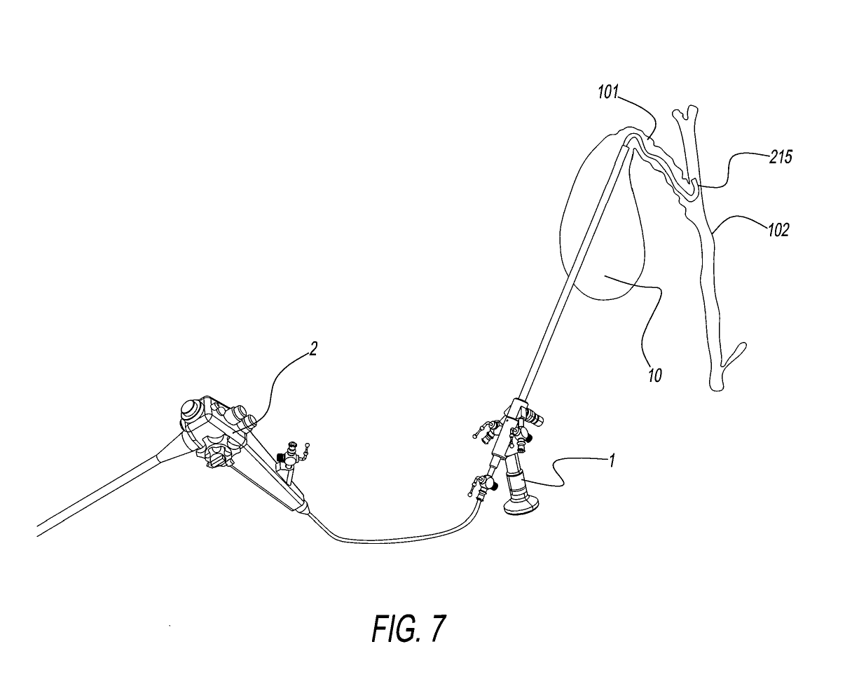

FIG. 7 is a schematic diagram of a mother-baby endoscope system consisting of hard mother-cholecystoscope and flexible baby-choledochoscope in surgery process. In the operation, a doctor determines the position of a gallbladder by placing a laparoscope into abdominal cavity of a patient and makes a minute incision at the bottom of gallbladder. And then the multi-operation-channel cholecystoscope 1 enters into the gallbladder cavity 10, the gallstones can be removed from gallbladder cavity 10. After finding the entry of cystic duct 101 via multi-operation-channel cholecystoscope 1, a flexible choledochoscope 2 is placed into cystic duct 101 through a wide instrument channel 16 of the multi-operation-channel cholecystoscope 1. Taking advantage of the front end with a small diameter and flexibility, the flexible choledochoscope 2 enters into the common bile duct 102 and left and right hepatic duct in porta hepatis, and small surgical instruments (such as basket type grasping forceps, biopsy forceps, etc.) can enter into an instrument channel connector 22 of the flexible choledochoscope 2 and conduct the surgical treatment.

1. A mother-baby endoscope system consisting of hard mother-cholecystoscope and flexible baby-choledochoscope, wherein said system comprises a multi-channel cholecystoscope utilized as mother endoscope and a flexible choledochoscope utilized as baby endoscope; said multi-channel cholecystoscope comprises a hard endoscope body, an instrument channel disposed at the hard endoscope body, an input port of an ocular, an input port of cold light source, a water inlet channel and a water outlet channel, and further comprises a light source host machine connected to said input port connector of cold light source of the mother endoscope, and a video camera system of the input port of the ocular; said flexible choledochoscope comprises a bendable front end, an instrument channel connected to said front end, an operating part, a light source host machine connected to said operating part via a cable, and a video camera system; a water supply equipment is also connected with said flexible choledochoscope for supplying water thereto; the front end of said baby endoscope passes through the instrument channel of said mother endoscope and wherein a bending portion is provided at an apex part of the front end of the flexible choledochoscope, and a bending angle of the bending portion is up to 180° in vertical direction, and up to 160° in horizontal direction, and a length of the bending portion is less than or equal to 25 mm.

2. (canceled)

3. The mother-baby endoscope system of claim 1, wherein the diameter of the apex part of the front end of the flexible choledochoscope is less than or equal to 3.0 mm, and the length of the apex part is 500 mm.

4. The mother-baby endoscope system of claim 3, wherein the flexible choledochoscope can be either an electronic choledochoscope which adopts an electronic imaging optical system, or a fiber choledochoscope which adopts a fiber imaging optical system.

5. The mother-baby endoscope system of claim 1, wherein the diameter of the front end of the hard endoscope body of the multi-channel cholecystoscope ranges from 6.5 to 7.5 mm, the length ranges from 200 mm to 220 mm.

6. The mother-baby endoscope system of claim 5, wherein the diameter of instrument channel of the multi-channel cholecystoscope is more than or equal to 3.5 mm.

7. The mother-baby endoscope system of claim 6, wherein said input port of the ocular of the multi-channel cholecystoscope utilized as a mother endoscope is at the angle of 135 degree to the central axis of hard endoscope body, which forms overall gun-shape; 2.8 mm optical lens are adopted by the optical system in the endoscope; There are three kinds of multi-channel cholecystoscopes classified by the optical lens having the field angle of 0 degree, 12 degree or 30 degree respectively.

8. (canceled)
